(19) United States
(12) Patent Application Publication
(10) Pub. No.: US 2015/0141871 A1
(43) Pub. Date: May 21, 2015

(54) PAIN MINIMIZING INJECTION SYSTEM

(71) Applicant: Kaveh KHAST, La Jolla, CA (US)

(72) Inventor: Kaveh KHAST, La Jolla, CA (US)

(21) Appl. No.: 14/494,517

(22) Filed: Sep. 23, 2014

Related U.S. Application Data
(60) Provisional application No. 61/881,852, filed on Sep. 24, 2013.

(51) Int. Cl. A61B 5/151; A61B 5/145; A61B 5/15

(52) U.S. Cl. CPC A61B 5/151; A61B 5/150137; A61B 5/14532
USPC 600/583; 600/365

(57) ABSTRACT

The present invention recognizes that there is a need for a single-step, pain-free or substantially pain-free device to test the levels of an analyte in one's blood such as glucose. The invention incorporates a lancet and a glucose test strip into a disposable cartridge. The user inserts the cartridge into the port of the device to turn it on. The base vibrates to quench or reduce pain from a lancet puncture. The test strip is exposed to the blood sample at the site of the lancet puncture which is analyzed by the device.

The present application claims priority to U.S. Provisional Application Ser. No.: 61/881,852, filed Sep. 24, 2013, which is incorporated by reference herein in its entirety.

### TECHNICAL FIELD

The present invention relates generally to blood lancets and injection systems, and, more particularly, to a single-step, relatively or substantially pain free or reduced pain blood lancet and blood sugar testing system.

### BACKGROUND

Diabetes is a common ailment often experienced as a result of insulin deficiency (Type 1) or insulin insensitivity (Type 2). An estimated 382 million people have diabetes worldwide, 90% of which are Type 2. The incidence of Type 2 diabetes has grown substantially with industrialization and the availability of refined sugars and processed foods. Blood sugar levels which are too high (hyperglycemia) or too low (hyoglycemia) can be dangerous.

Regular monitoring of blood glucose levels is essential to reducing diabetes associated complications. Both type 1 and type 2 diabetes subjects and patients may be forced to regularly monitor their blood glucose levels using a lancet device and an electronic blood glucose testing device. For the typical diabetic, it may be necessary to check his or her blood glucose level four to six times per day.

However, many people have an aversion to medical lancets, syringes and the sight of blood, even those who must regularly take blood samples. These issues, along with the time and effort necessary for testing may cause people to avoid doing so. Without regular testing, subjects and patients may face unsafe levels of glucose and emergency situations. High levels of glucose may lead to ketoacidosis which is a life-threatening condition which needs immediate treatment. Further, diabetics may experience a myriad of long-term health issues related to chronic hyperglycemia including blindness, vascular disease, loss of limbs, and kidney failure.

Conventional glucose testing requires the use a relatively painful and large lancet to draw a blood sample from one's skin. A spring-activated device which ejects a lancet is often used to make the size of the puncture in the skin more predictable. A small amount of blood will ooze out of the skin at the sight of the puncture. The user may have to squeeze the area to increase the amount of blood for testing. The next step is physically contacting the blood sample with the end or an application zone of a glucose test strip. Then the test strip is inserted into an electronic or other type of glucose monitor which displays the results value, such as concentration of glucose in the sample.

This process involves several steps using multiple devices with usually uniquely compatible lancets and test strips. The physical pain involved is also an apparent issue, particularly when fingertips must be repeatedly lanced. Further, people may have an aversion to the sight of blood. For these reasons, an improved method of blood testing is necesary. None of the conventional systems have the attributes of the present invention.

For example, U.S. Pat. No. 579,294 issued Jan. 18, 1994 to Anderson, refers to a portable medical diagnostic system which includes a lancet device and a microprocessor in one device. Although the device combines a lancet with an analyzer, the user must be supplied with separate components and partake in multiple steps for a blood analysis. The user must insert a lancet tip into the device to puncture the skin. This requires a conventional lancet puncture which is also painful. The user must produce a separate test strip and expose it to the blood. In contrast, the present invention is directed to a single-step blood test which does not require multiple components and is substantially pain-free.

### SUMMARY

The present invention recognizes that there exists a long felt need for a device which incorporates both a lancet, a glucose monitor and a vibrating base to yield a one-step, substantially pain-free or reduced pain glucose analysis device.

A first aspect of the present invention is a base assembly with a glucose analysis computer and display monitor of the present invention.

A second aspect of the present invention is a base assembly including a vibrating base of the present invention.

A third aspect of the present invention is a base assembly including an opening or port at the distal end for a lancet cartridge of the present invention.

A fourth aspect of the present invention is a disposable lancet cartridge of the present invention.

A fifth aspect of the present invention is a disposable lancet cartridge with both an internal pressure or spring-release lancet and an internal glucose (or other analyte) test strip of the invention.

A sixth aspect of the present invention is a disposable cartridge wherein the test strip is located sufficiently close to the lancet tip such that the user does not have to visualize the blood sample for analysis.

A seventh aspect of the present invention is a disposable lancet cartridge wherein the test strip is located sufficiently close to the lancet tip such that the blood sample is attracted to the test strip by capillary action.

A eighth aspect of the present invention is a disposable lancet cartridge wherein a lancet tip is ejected and quickly detracted for safety.

An ninth aspect of the present invention is a method of using the vibrating base of the invention to obtain a blood sample from the skin or mucousal tissue while inflicting no or minimal pain.

A tenth aspect of the present invention is a method of using the vibrating base of the invention to test one's blood glucose level (or other analyte) while inflicting no or minimal pain.

A eleventh aspect of the present invention is a method of using the vibrating base of the invention to inject a medicinal substance into the skin or mucousal tissue while inflicting no or minimal pain.

### BRIEF DESCRIPTION OF THE FIGURES

FIG. 1 generally depicts a front view of the base assembly (1) of the invention. The base assembly can include a screen (3) to display data including the user's blood glucose level. The outside surface of the base assembly can be a composed of sturdy material with a surface that offers durability and may be water proof. Ridges (2) can be included on the handle of the base assembly for improved ergonomics.

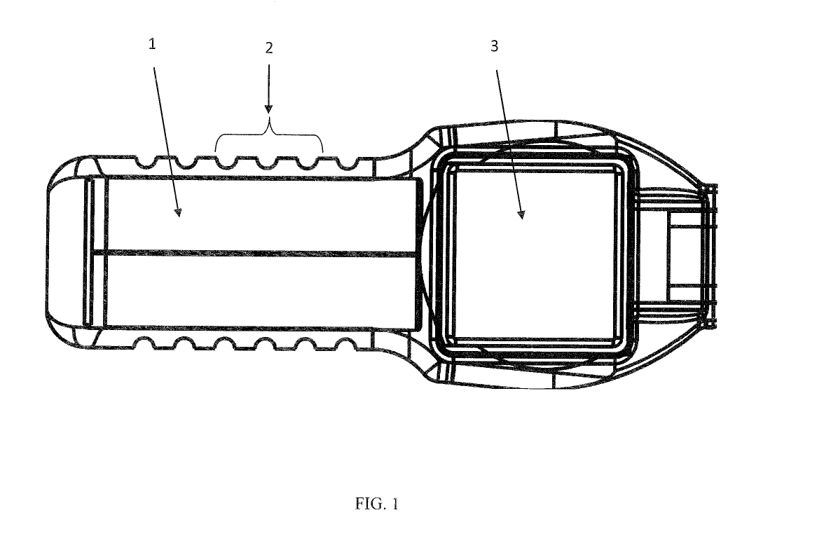

FIG. 2 generally depicts a bottom view of the base assembly (1) of the invention. The system is turned “on” when the user inserts a lancet cartridge into the port at the distal end (4). The interior of the base near the port (5) can house a motor which drives an off-set weight (not shown).

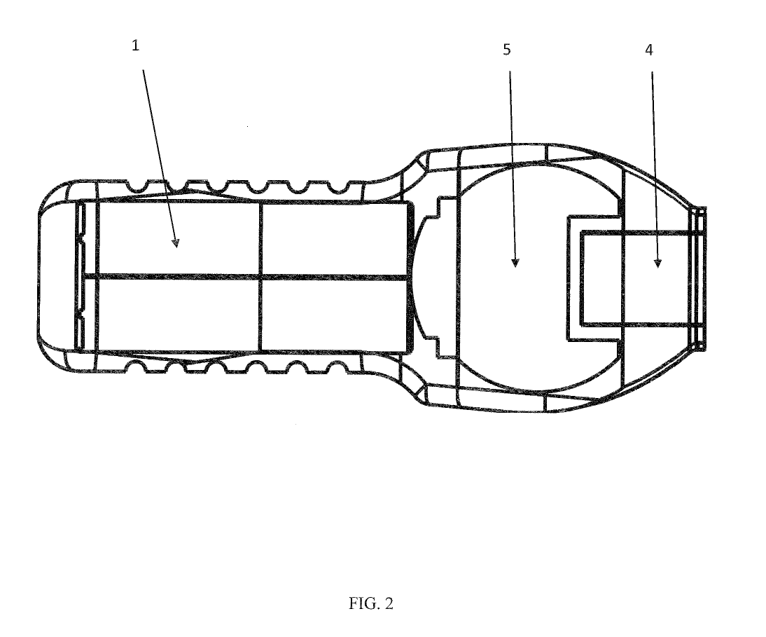

FIG. 3 generally depicts a bottom view of the base assembly (1) of the invention. The lancet cartridge (6) is present in the port at the distal end (4).

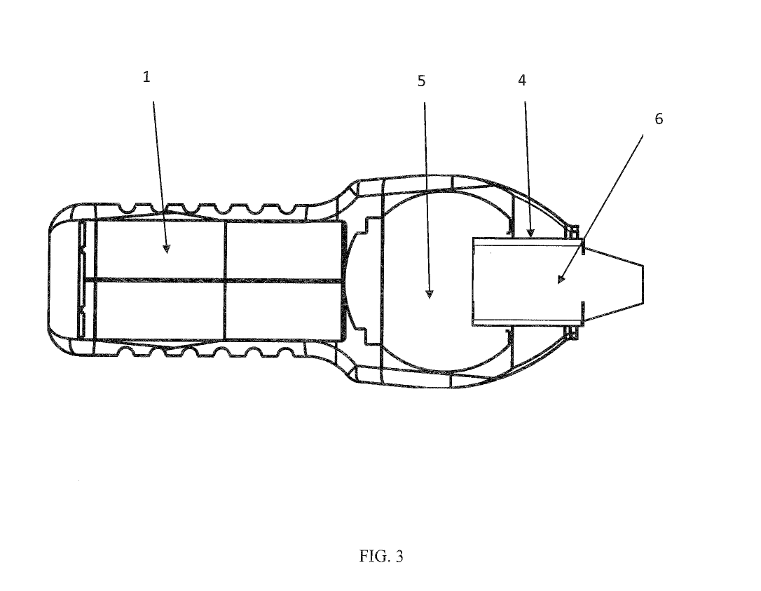

FIG. 4 generally depicts the screen (3). The screen may or may not be a “touch screen” which can also display the operations status as well as instructions on using the system, the date and time, the battery status, stored information related to the user(s) and information related to ailments including diabetes.

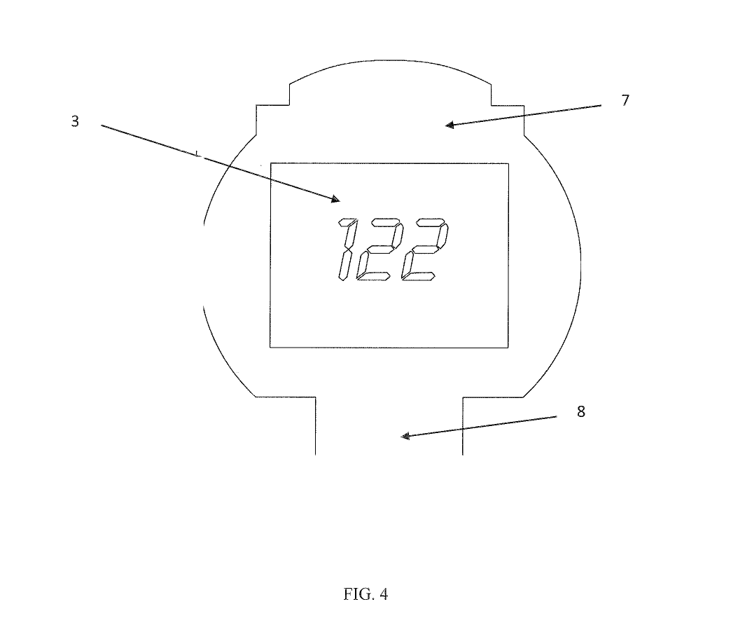

FIG. 5 generally depicts a perspective view of the lancet cartridge (6) with the bottom, open end (9). In this figure, the lancet (7) has been ejected and is positioned close to the test strip (8).

FIG. 6 generally depicts a cross-sectional view of the side of the cartridge. The cartridge includes both a lancet (7) and a test strip (8). The top of cartridge fits into the docking port of the base (not shown). After the cartridge snaps into place, the patient can remove the safety cover (13) from the cartridge. The lancet (7) is an appropriate gauge and length to produce a sufficient blood sample. A spring (10) is used first to eject the lancet. The test strip (8) is close to the site of the lancet tip to readily obtain a blood sample. The test strips may collect a sample of blood through a “window” or opening for the blood sample (14). Windows can be on both sides of the bottom of the test strip. The test strip can function with as little as 0.5 μl of blood, from either or both sides of the test strip. The test strip fits into an electromagnetic reader (11), and the test results are reported to the data processor (12) for storage.

The distance between both the lancet (and subsequent lancet puncture) and the test strip is about 0.5 mm. The test strip extends about 1.5 mm from the base of the cartridge when pressed against the skin. This proximity aids in collecting a sufficient and consistent blood sample without requiring the user to visualize the blood and move the test strip.

FIG. 7 generally depicts a cross-sectional view of the side of the cartridge. Here the lancet (7) is locked in place against the skin (20). The test strip (8) is in contact with the skin (20) and the tip of the test strip (14) can press or flex against the skin

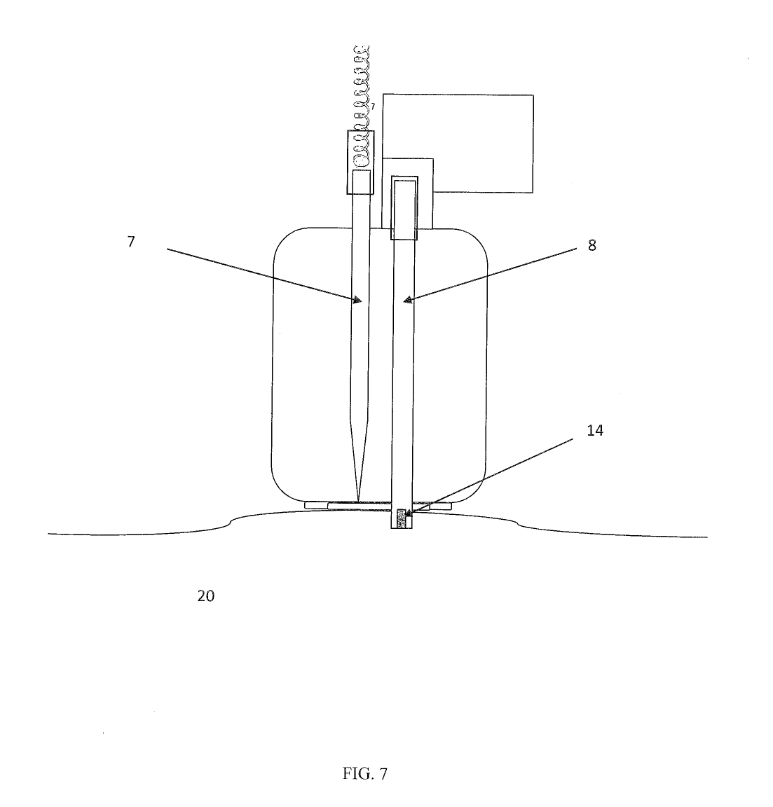

FIG. 8 generally depicts a cross-sectional view of the side of the cartridge. Here the lancet (7) has penetrated the skin and the test strip (8) is in contact with the skin (20) to contact a blood sample.

FIG. 9 generally depicts a bottom view of the lancet cartridge. The lancet (11) is seen in close proximity to the test strip (15).

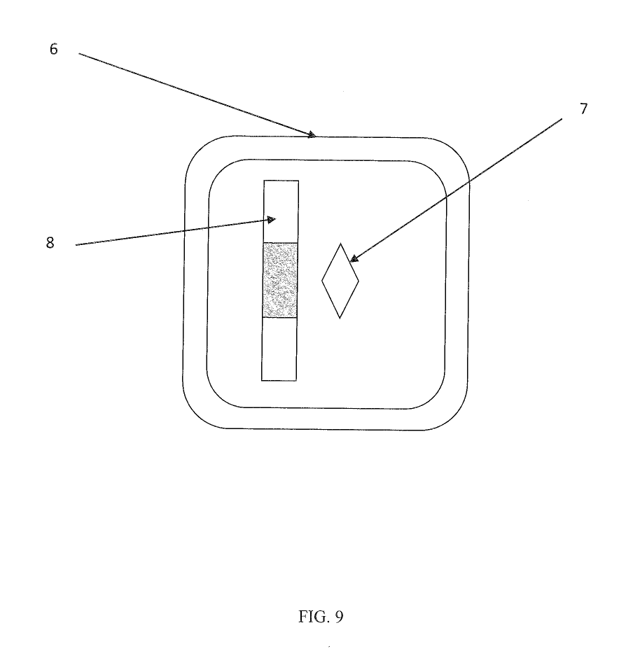

### DETAILED DESCRIPTION OF THE INVENTION

### Definitions

Unless defined otherwise, all technical and scientific terms used herein have the same meaning as commonly understood by one of ordinary skill in the art to which this invention belongs. Generally, the nomenclature used herein and the clinical procedures in medical care and diabetes in general, and other applicable technologies described below are well known and commonly employed in the art. Where a term is provided in the singular, the inventors also contemplate the plural of that term, and when a term is provided in the plural, the inventors also contemplate the singular of that term. The nomenclature used herein and the laboratory procedures described below are those well known and commonly employed in the art unless set forth otherwise. As employed throughout the disclosure, the following terms, unless otherwise indicated, shall be understood to have the following meanings:

While the invention is primarily described in diabetic applications, in particular in the context of glucose concentration measurements, it is understood that the present invention is not so limited and may be employed to measure any analyte or other substance in the blood. It may also be used a drug delivery application, particularly where the drug delivery regime is self-administered. Other applications include, for example, but not limited to, the administration of an immunization, asthma medication, blood thinners, insulin or steroid.

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” and “below” used herein are merely for ease of description and refer to the orientation of the components as shown in the figures. It should be understood that any orientation of the components described herein is within the scope of the present invention.

Other technical terms used herein have their ordinary meaning in the art that they are used, as exemplified by a variety of technical dictionaries.

### Introduction

The present invention recognizes that there exists a long felt need for a unitized blood glucose monitoring system that elicits little or no pain or other undesirable features.

Generally, the present invention is a one-step device for blood glucose testing which minimizes or reduces the pain caused by a lancet puncture. The device includes a handle or base which is activated by inserting a disposable cartridge. The handle of device vibrates against the user's skin before and/or during ejection of the lancet. The vibration causes nerves to carry the tingling feeling to the brain. Because the nerves are excited, their ability to transmit pain is impeded. Hence, The stimulation from the vibrator acts to mask the pain signals caused when the needle or the like penetrates the skin.

The disposable cartridge incorporates a glucose test strip along with a lancet. The handle triggers the release of the lancet to penetrate the user's skin. The test strip is located within a close distance to the site of the puncture, preferably less than one millimeter. This allows the test strip to automatically come into contact with the blood sample without the user's involvement. The tip of the test strip may use capillary action to attract the blood. Thereafter, internal electronics in the handle analyze the test strip and display the blood glucose level. The components of the cartridge, including the lancet, may be composed of nonconductive materials to maintain the integrity of electric signals transferred across the test strip.

Because the test strip is integrated into the lancet assembly, the user does not need separately handle a test strip thus previously preventing the sensor strip from being polluted by the user's hands It is also more convenient for the user to purchase cartridges rather than lancets and test strips separately for different devices.

As a non-limiting introduction to the breath of the present invention, the present invention includes several general and useful aspects, including:

1) A base assembly which is ergonomically shaped.

2) A base assembly including a vibrating shaft of the present invention to reduce pain from a lancet or syringe.

3) A base assembly with a switch or pressure-activated vibrating mechanism of the present invention.

4) A base assembly of the present invention which includes internal electronics, a microprocessor, data memory storage and a monitor to analyze a test strip and display a reading.

5) A lancet cartridge of the present invention which is disposable and intended for single use.

6) A base assembly of the present invention which is turned on when a lancet cartridge is inserted into the port or opening on the bottom or distal end.

7) A lancet cartridge of the present invention which includes a spring or pressure activated lancet.

8) A lancet cartridge of the present invention which includes an internal test strip that collects blood at or near the site of a the puncture from the lancet tip via proximity or capillary action.

9) A lancet cartridge of the present invention which includes a test strip that uses an enzyme specific to glucose or other analyte for analyzing a blood sample to determine its level of glucose or other analyte.

10) A method of using the base assembly of the present invention with the lancet cartridge to reduce or abrogate pain inflicted by a lancet or needle.

11) A method of using the base assembly with the lancet cartridge of the present invention to obtain a blood sample while inflicting minimal or no pain.

12) A method of using the base assembly with the lancet cartridge of the present invention to determine a subject's blood glucose level or other analyte.

13) A method of using the base assembly with the lancet cartridge of the present invention to inject a medicinal or other substance into a subject's blood stream.

These aspects of the invention, as well as others described herein, can be achieved by using the methods, articles of manufacture and compositions of matter described herein. To gain a full appreciation of the scope of the present invention, it will be further recognized that various aspects of the present invention can be combined to make desirable embodiments of the invention.

I BASE ASSEMBLY

A first aspect of the present invention is a base assembly (1) that includes ridges (2) for an improved ergonomic grip, a port (4) for a single-use lancet cartridge (6), an internal blood glucose analyzer (not shown) and a screen (3) to display data including the subject's blood glucose level or level of other analyte(s). The screen may be a “touch screen” which can also display various information including instructions on using the system, the date and time, the battery status, stored information related to the user(s) and information related to ailments including diabetes. Data related to the user's exercise and diet may also be entered manually or by syncing with another device. The base assembly may analyze data and the screen may also post notifications or reminders to the user to organize and encourage regular glucose testing.

The base assembly remains in “stand by” mode without a lancet cartridge. To turn the base assembly “on” a patient inserts a cartridge into the opening at the bottom of the base assembly. (8) This puts the system into “active” mode. Because test strips may vary from batch to batch, the cartridge and/or test strip may contact a code to accurately calibrate it with each cartridge and/or test strop. By entering the coding or chip into the glucose meter, the meter will be calibrated to that particular cartridge and/or test strip.

The base assembly also contains a vibrating mechanism located near the port in the distal area of the base. (5) The vibrating is activated by a switch or automatically when the bottom of the base assembly is pressed against a user's or patient's skin. The entire device, including the cartridge may vibrate. However, the vibration is most pronounced at the bottom, where it comes into contact with the skin. As the device is pressed against the patient's skin, vibrations are transmitted to the skin of the patient or subject.

Under the “pain gate” theory the nerves at the location are temporarily desensitized to pain. This occurs because both thin and large diameter nerve fibers carry information from the lancet site to two destinations in the spinal cord:

1) transmission cells that carry the pain signal to the brain and

2) inhibitory interneurons that impede transmission cell activity. Thin fiber activity impedes inhibitory cells while large diameter fibers activity excites the inhibitory cells. Thin fibers are activated by pain while large diameter nerve fibers are activated by touch, pressure and vibration. So with more large fiber activity (pressure and vibration) relative to thin fiber activity at the inhibitory cell, less pain in felt by the subject. This may explain why it is common for a person to rub the site of an injury.

The lancet cartridge is activated instantly or after a short delay of up to a few seconds as the nerves are exposed to the pressure and/or vibrations of the base assembly (1). The user may adjust the level of vibration using a dial or by programming the unit using an “options” menu on a touch screen. (3) The vibration may be turned off or may be increased to a greater intensity and frequency to satisfy the user's desires. Another use of the base assembly may be as a massaging device. The source of the vibration can be a small motor that spins an offset weight. (not shown)

In an alternative setting, the user activates the vibrating motor with gentle pressure of the base against the skin. The user feels the tingling. Heavier pressure activates the cartridge to release the lancet. This allows the user to control both the vibrating phase and the lancet release phase by simply varying the pressure.

Because the energy source and the circuitry of the device may be electromagnetic, it can be necessary to shield interior components from electromagnetic static. Electromagnetic concerns are addressed by isolating the vibrator chamber, shielding the other electromagnetic static and grounding of the entire system.

The walls create an isolated chamber and shield the other elements from stray electromagnetic static that may be generated by the motor. An appropriate metal plate such as titanium alloy may act as a shield. Shields for this invention can be made of any appropriate material that can isolate and protect against stray energy from any energy-producing source, including but limited to: electromagnetic, photovoltaic, AC/DC current, and nanotechnological structures and/or layers. For example, the motor can be covered with a bendable metal (titanium alloy) strip, wrapped about the cylindrical portion of the motor. This element can help to stop electromagnetic static at the source. Note that the entire electromagnetic system is grounded

The base assembly can also include a port for charging internal batteries (not shown) and a universal serial bus (USB) port or other similar port for connecting and/or syncing to a computer or other device. While the base assembly may store data from its user, it may be synced with other devices and computers to maintain records of glucose levels. This may also be done wirelessly by utilizing blue-tooth, internet or cell phone technology. The data may also be relayed to a nurse, dietician, counselor, physician, parent, care-provider or other party to improve diabetes management and/or diet.

A computer in the base assembly may be programmed to assist the user in various ways including:
instructing the user on how to use the device,
recording blood glucose levels, meals and exercises,
encouraging the user to exercise, refrain from eating glucose or make other lifestyle suggestions,
calculating and recording glucose spikes and taking steps to prevent such,
assisting with diabetes management by reminding the user when to test and
calculating insulin dosages and referring the user to see his or her health care provider when necessary.

Data and information is displayed on the monitor and/or transmitted audibly. The base assembly may include an audio speaker to audibly communicate the values to the user. The speaker may also be used to play noises, music or alarms to organize and remind the user to test and/or take food or medication. The base assembly may also be used to play music files (e.g. mp3) using the speaker or a headphone jack (not shown).

II CARTRIDGE

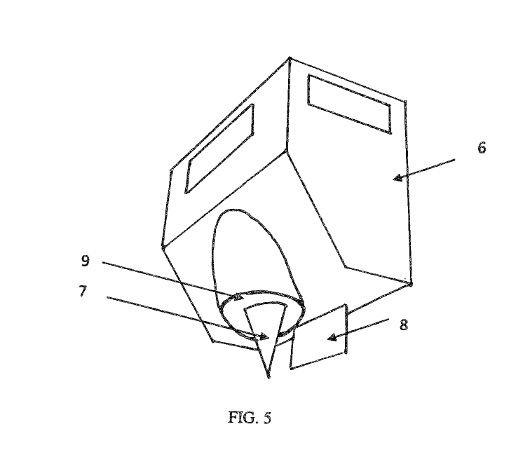

Another aspect of the invention is a lancet assembly (FIG. 5) for puncturing the skin to get a blood sample composed of a housing having at least one open end (9), a lancet (7) with a lancet tip stored inside a housing (6), a triggering device for ejecting said lancet from said at least one open end (not shown), a lancet track means for guiding said lancet on a predetermined puncture path so that said lancet tip is expelled out of the open end, a locking device to allow lancet to remain in locked position, a triggering mechanism (not shown) to eject the lancet (7) and a test strip (8)for analyzing glucose or other analyte.

The lancet cartridge contains the lancet (7) which is ejected by a spring (10) or other source of pressure. The lancet is ejected a pre-set distance, preferably about one to five micro-meters. The user may choose between lancet cartridges which eject the lancet at various lengths. Lancet assemblies may be available for use on finger tips as well as alternate locations (e.g. palms, thighs or upper arms). In the alternative, the cartridge may have a means to allow the user to adjust the distance of ejection. This might be a dial on the base or by programming the system using the touch screen.

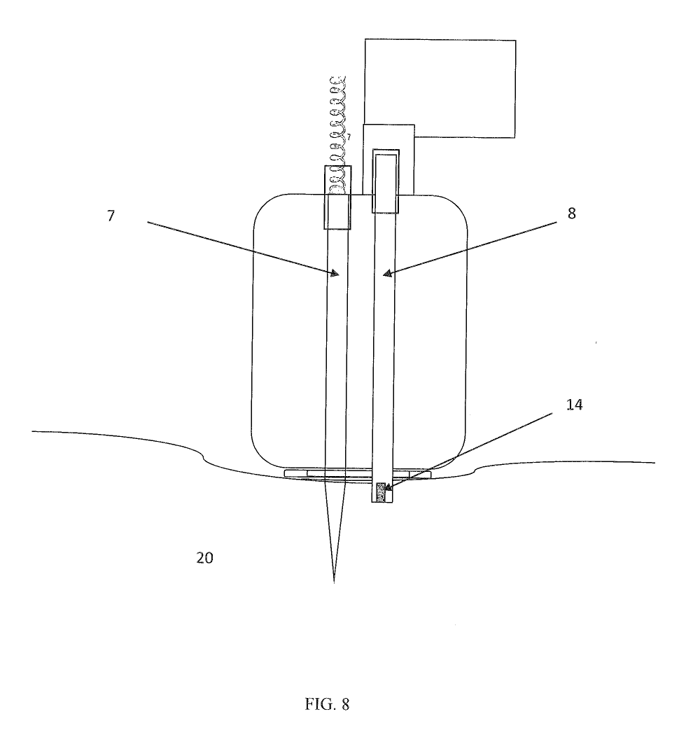

It will be understood that this type of cartridge and lancet system is only exemplary and other types of cartridges can be used. In this type of cartridge the lancet and lancet body are spring-based and are contained within a housing. The lancet body is held in place by two hooks that are held in notches. (not shown) When the device is pressed against a patient's skin, the ramps press against the hooks due to the inclined ramp relationships press the hooks outwardly and out of the notches, thereby allowing the spring to push the lancet forward and into the patient's skin. (FIG. 8)

Upon getting triggered by the base assembly, the lancet (7) is ejected from the lancet assembly. (6) The tip of the lancet enters the skin to cause a puncture which produces a blood sample. The lancet tip is quickly retracted back into the case of the lancet assembly to prevent injury and exposure to the user and others. Thus, the user does not have to physically handle the used lancet tip.

A test strip (8) is integrated into the lancet cartridge (6). The test strip may be made of flexible material and/or get released from the cartridge in unison with the lancet. Blood from the puncture will come into contact with the tip or edge of the test strip. (14) Capillary action may assist in attracting blood to the test strip. The tip of the test strip may be shaped to improve capillary action. Because of this and the proximity, the user does not have to move the device or visualize the blood sample.

The test strip may (14) use an electrochemical or amperometric method to determine the level of glucose in a sample. The measurement assembly (12) is activated when the tip of the test strip contacts a sufficient volume of blood. The measurement assembly relays data and values to a digital screen which displays the measured value of the glucose or other analyte. The level should be displayed within a few seconds of reading the sample. A speaker can audibly communicate the value to the user. (not shown)

A further aspect of the invention, the lancet assembly including the lancet and internal parts are composed of nonconductive material. This helps maintain the integrity of electric signals that are transferred across the test strip during blood testing. This avoids inaccurate results or other problems associated with electromagnetic fields. In the alternative, the cartridge may be surrounded by magnetic material to “quench” magnetic fields that may interfere with electronic transmission across the glucose strip.

III METHOD OF USING A BASE ASSEMBLY AND CARTRIDGE TO ANALYZE

Another aspect of the present invention is a method of using a base assembly (1) and cartridge (6) of the present invention, including: a) providing a patient or user; b) providing a base assembly of present invention; c) providing a lancet cartridge of present invention; d) inserting the lancet cartridge into the port (4) of the base assembly; e) pressing the bottom of the base assembly against the skin of a subject; f) utilizing vibrating action of the base assembly to mask or distract pain signal nerve; g) triggering the release of the lancet from the lancet cartridge to produce a puncture and blood sample; h) allowing the test strip of the lancet cartridge to contact the blood sample; and i) determining the level of glucose or other analyte in the blood sample.

The subject can be any animal, including non-mammals, mammals, primate and non-primate, non-human primates, and humans. Humans are preferable, but are not necessary. Smaller drop volumes have enabled “alternate site testing.” Blood samples can be obtained at the upper arm, thigh, calf or other less sensitive areas instead of the fingertips.

IV METHOD OF USING A BASE ASSEMBLY AND CARTRIDGE TO INJECT

Another aspect of the present invention is a method of using a base assembly and cartridge of the present invention. The cartridge can contain a syringe (not shown) with a medicinal such as a drug to be injected into the user or patient. This aspect includes: a) providing a patient or user; b) providing a base assembly (1) of present invention; c) providing a cartridge (6) of present invention with an internal syringe; d) inserting the lancet cartridge into the port (4) of the base assembly; e) pressing the bottom of the base assembly against the skin of a subject; f) utilizing vibrating action of the base assembly to mask or distract pain signal nerve; g) triggering the release of the syringe from the cartridge to produce a puncture and blood sample; h) allowing a substance in the syringe to enter into the blood skin of the individual. When the injection is complete, the device will indicate such to the user audibly or on the screen.

The subject can be any animal, including non-mammals, mammals, primate and non-primate, and humans. Humans are preferable, but are not necessary. Smaller drop volumes have enabled “alternate site testing.” Blood sample may be obtained at the upper arm, thigh, calf or other less sensitive areas instead of the fingertips.

### EXAMPLES

### Example 1

### Using the Technology

Pain Free Glucose Test

This example provides for and establishes the early development of the technology and prototype base, lancet cartridge, methods of making same, and methods of using same.

The user checks the screen on the base to begin The screen indicates the operational status of the device, including the status of the batteries and whether the device is ready for use. The user may also follow instructions that can be displayed on the screen.

The user may need to charge the unit by inserting a charger into a charging port (not shown) or inserting batteries into the unit.

The user will then obtain a lancet cartridge. The lancet cartridge is sterile and intended for single use. Accordingly, the user will remove the packaging and/or cover of the lancet cartridge. This will expose the bottom of the cartridge with the test strip.

The user then inserts the cartridge into the docking port of the base of the assembly. Arrows on the cartridge may be present (not shown) to indicate the proper placement to the user. The top of the cartridge has electrical contacts that configure and/or sync with a computer or circuit board in the base. The bottom of the cartridge has a test strip and an opening for ejection of the lancet tip. The assembly is turned “on” when the cartridge is inserted into the port and the screen will indicate whether the device is ready for use. The user may also refer to the screen for additional instructions.

The user may then touch the base to his or her skin. Light pressure on the base of the device will activate the vibrating motor. The entire base may vibrate but the vibrations are most pronounced at or around where the lancet cartridge is located. The user will feel the tingling sensation on his or her skin. This may “confuse” the nerves or activate nerve fibers that will impede the transmission of pain.

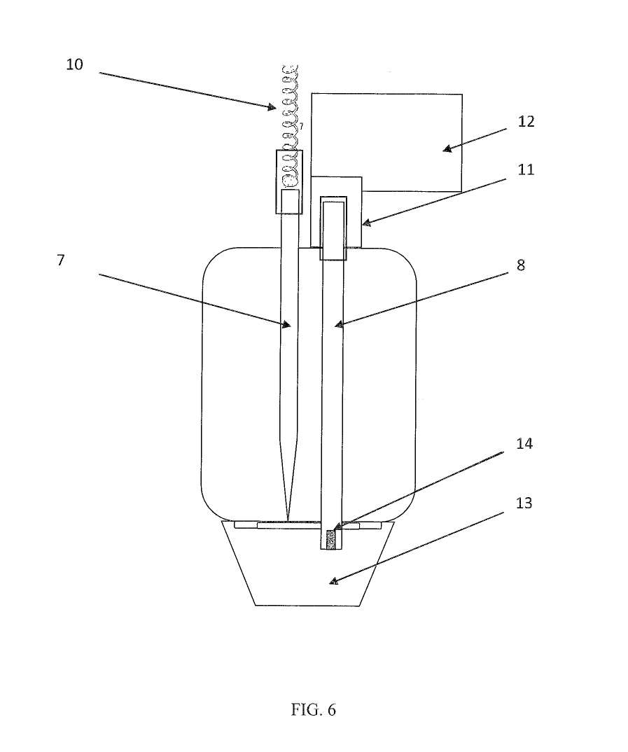

As the user presses the device against the skin, the lancet cartridge is activated (FIG. 6). This may occur instantaneously or after a short delay, according to how the unit is programmed The lancet is ejected so that the tip enters the skin of the user to cause a puncture at or near the test strip. The user will feel little or no pain because of preceding vibrations.

The user should continue to hold the base against his or her skin and look at the screen. The test strip will contact with the blood sample and the device will measure the electrical current across the test strip. This is used to calculate the blood glucose level in the blood sample. Upon determining the level of glucose, the device will beep or ring. The screen will display the value and may provide other relevant information to the user, including instructions or suggestions on further testing and/or diabetes management. The glucose level will be stored along with the date and time and may be transferred or synced with a computer or other device.

If the device is unable to determine the level of glucose, the screen will indicate potential problems. It will instruct the user to remove the lancet cartridge and repeat the process. This may occur if the lancet puncture does not produce an adequate volume of blood or if the user moves the device away from the blood too quickly.

Further refinements are made and further prototypes and preferred embodiments developed. The product depicted in the figures result from such efforts.

Pain Injection

In this example, the invention is used to inject a substance such as a steroid, immunization, insulin or other medicinal substance into the patient or subject with little or no pain.

The user checks the screen on the base to begin. The screen indicates the operational status of the device, including the status of the batteries and whether the device is ready for use. The user may also follow instructions that can be displayed on the screen.

The user will then obtain a cartridge that is equipped with a particular injectable substance. The cartridge is sterile and intended for single use. Accordingly, the user will remove the packaging and/or cover of the cartridge.

The user then inserts the cartridge into the docking port of the base of the assembly. Arrows on the cartridge may be present (not shown) to indicate the proper placement to the user. The top of the cartridge has electrical contacts that configure and/or sync with a computer or circuit board in the base. The bottom of the cartridge has a test strip and an opening for ejection of the tip of a syringe. The assembly is turned “on” when the cartridge is inserted into the port and the screen will indicate whether the device is ready for use. The user may also refer to the screen for additional instructions.

The user may then touch the base to his or her skin. Light pressure on the base of the device will activate the vibrating motor. The entire base may vibrate but the vibrations are most pronounced at or around where the cartridge is located. The user will feel the tingling sensation on his or her skin. This may “confuse” the nerves or activate nerve fibers that will impede the transmission of pain.

As the user presses the device against the skin, the cartridge is activated. This may occur instantaneously or after a short delay, according to how the unit is programmed. The tip of the syringe is ejected so that the tip enters the skin of the user. The user will feel little or no pain because of preceding vibrations.

The user should continue to hold the base against his or her skin and look at the screen. Upon completing the injection, the device will beep or ring. The screen will display an “okay” message and may provide other relevant information to the user, including instructions or suggestions on further injections.

Further refinements are made and further prototypes and preferred embodiments developed. The product depicted in the figures result from such efforts.

All publications, including patent documents and scientific articles, referred to in this application and the bibliography and attachments are incorporated by reference in their entirety for all purposes to the same extent as if each individual publication were individually incorporated by reference.

All headings are for the convenience of the reader and should not be used to limit the meaning of the text that follows the heading, unless so specified.

What is claimed is:

1. A blood diagnostic device which produces a pain-less or near pain-less lancet puncture composed of:
a) a port for a lancet cartridge,
b) a lancet cartridge with an internal lancet and internal test strip,
c) a motor to cause physical vibration at or near the at least one open end or port and
d) a means of ejecting said internal lancet from said lancet cartridge to cause a puncture in the skin and produce a blood sample for analysis.

2. The blood diagnostic device of claim 1 wherein said internal test strip measures an analyte such as glucose in the blood.

3. The blood diagnostic device of claim 1 which includes a digital monitor to display the measured value of an analyte such as glucose to the user.

4. The blood diagnostic device of claim 1 wherein said lancet cartridge is disposable or single use.

5. The blood diagnostic device of claim 1 wherein the user activates said physical vibration by pressure against the base of the device against a patient's or subjects skin or tissue.

6. The blood diagnostic device of claim 1 wherein the user activates said lancet cartridge by pressure against the base of the device against a patient's or subjects skin or tissue.

7. The blood diagnostic device of claim 1 wherein the tip of said internal lancet retracts after ejecting.

8. The blood diagnostic device of claim 1 wherein said internal test strip uses capillary action to contact a blood sample.

9. The blood diagnostic device of claim 1 wherein the user can adjust the distance that said internal lancet is ejected from said lancet cartridge.

10. The blood diagnostic device of claim 1 wherein said measured value is recorded digitally or relayed to a computer or other electronic device.

11. The blood diagnostic device of claim 1 wherein a said lancet cartridge includes a syringe.

12. The blood diagnostic device of claim 1 which includes a syringe for withdrawing blood from a human or animal for diagnostic purposes.

13. The blood diagnostic device of claim 1 which includes a syringe for injecting a drug, steroid, insulin or immunization solution into a human or animal.

14. A method of measuring blood glucose levels in a human or mammal by inflicting little or no pain using the blood glucose meter device of claim 1, comprising the steps of:
a) activating the device to vibrate against the skin of a subject or patient,
b) triggering said lancet eject to puncture the skin and produce a blood sample,
c) allowing said test strip to contact said sample of blood,
d) displaying the concentration of an analyte value such as glucose to the user on a screen or audibly with a speaker.

15. The method of claim 14 wherein said blood glucose meter device is turned on by inserting a lancet cartridge into the base or port.

16. The method of claim 14 wherein said lancet assembly is intended for single use or disposable.

17. The method of claim 14 wherein said test strip is positioned less than five millimeter asay from the tip of said lancet.

18. The method of claim 14 wherein said test strip is ejected with or after said lancet tip is expelled out of the open end.

19. The method of claim 14 wherein said test strip uses capillary action to contact a blood sample.

20. The method of claim 14 wherein the user may adjust the distance that said internal lancet is ejected from said lancet cartridge.

21. The method of claim 14 wherein said lancet device is used for withdrawing blood from a human or animal for diagnostic purposes.

22. The method of claim 14 wherein said lancet cartridge contains a syringe for withdrawing blood.

23. The method of claim 14 wherein said lancet cartridge contains a syringe for injecting a substance such as an analyte, drug, steroid, insulin or immunization into a subject.

24. A lancet assembly for making a skin puncture and a resulting blood sample comprising:
a) a housing with a spring or pressure loaded lancet,
b) said housing having at least one open end,
c) a triggering device for ejecting said lancet from said at least one open end and
d) a test strip for analyzing an analyte such as glucose.

25. The lancet device of claim 24 wherein said lancet device is sterile and intended for single use or disposable.

26. The assembly of claim 24 wherein said lancet assembly comprises nonconductive material.

27. The assembly of claim 24 wherein said test strip is ejected with or after said lancet tip is expelled out of the open end.

28. The assembly of claim 24 wherein said test strip uses capillary action to contact a blood sample.

29. The assembly of claim 24 wherein said triggering device may be adjusted to increase or decrease the distance that it is ejected from said housing.
